(19) United States
(12) Patent Application Publication
(10) Pub. No.: US 2018/0092659 A1
(43) Pub. Date: Apr. 5, 2018

(54) SINGLE DRIVING BUTTON CONTROL-TYPE ULTRASONIC SURGICAL APPARATUS AND METHOD FOR CONTROLLING ULTRASONIC OUTPUT USING SAME

(71) Applicant: Ligasonix, Inc., New York, NY (US)

(72) Inventor: Eungkook Kim, Seoul (KR)

(21) Appl. No.: 15/539,095

(22) PCT Filed: Dec. 22, 2015
(86) PCT No.: PCT/IB2015/002598

§ 371 Date: Sep. 14, 2017

(30) Foreign Application Priority Data
Dec. 22, 2014 (KR) 10-2014-0186224

(51) Int. Cl. A61B 17/32; A61B 17/22

(52) U.S. Cl. CPC A61B 17/320092; A61B 2017/00017; A61B 17/22012

(57) ABSTRACT

The present invention relates to an ultrasonic wave surgery apparatus controlled by a single switch button and a control method of ultrasonic power using the same, where the an ultrasonic wave surgery apparatus controlled by a single switch button comprises: a body with an accommodating space therein, comprising: a grip that can be gripped by the user, and; an ultrasonic wave delivery unit which extends forward by a distance to allow for access to tissue; an transducer unit that is disposed in the accommodating space and which causes oscillations; an ultrasonic transducer unit disposed at the front end of the ultrasonic wave delivery unit and which comprises an end effector that is connected to the transducer unit and receives transmission of oscillations; a gripping unit disposed at the front end of the ultrasonic wave delivery unit and formed to allow variation of the distance thereof from the end effector, comprising an operating portion on the body to allow for control of the gripping portion; a start button disposed on the body and which causes an input signal according to controls by the user, and; a control portion which, if an input signal from the start button occurs once within a preset reference time duration and is sustained, operates the transducer unit at a first oscillation strength, and which, if an input signal from the start button occurs n times within a preset reference time duration and is sustained, operates the transducer unit at an n^{th} oscillation strength.

### TECHNICAL FIELD

The present invention relates to an ultrasonic wave surgery apparatus controlled by a single switch button and control method of ultrasonic power using the same. More specifically, the present invention relates to an ultrasonic wave surgery apparatus controlled by a single switch button to reduce operational fatigue and allow for intuitive control, and a control method of ultrasonic power using the same.

### BACKGROUND ART

Normally, a variety of ultrasonic wave surgery apparatuses are being used to cut tissue or carry out procedures such as cautery (coagulation).

In conventional cases, the operation of the transducer units of ultrasonic wave surgery apparatuses was carried out using foot pedal-type operating devices. In this method, the user, while using the ultrasonic wave surgery apparatus, operates the start buttons using his/her feet. Such foot pedal-type operating devices require the user to continually move the lower portion of his/her body, greatly undermining the stability with which procedures are carried out and leading to numerous cases where the operation of buttons is confused when using together with other devices, causing much discomfort to users.

To solve this problem, in recent times, ultrasonic wave surgery apparatuses with start buttons on the hand piece so that they can be held by the user have been developed. Accordingly, the user, gripping the handle, normally uses one finger to control the switch buttons.

However, ultrasonic wave surgery apparatuses in this form are equipped with a plurality of start buttons to allow for the setting of the strength of the ultrasonic wave power from the transducer unit to various levels. Accordingly, cases wherein users confused the operation of buttons still frequently occurred, and as a plurality of buttons was being controlled while gripping the handle, fatigue in the hand increased rapidly.

Accordingly, a method to solve these problems is called for.

### DETAILED DESCRIPTION OF THE INVENTION

### Technical Problem

The purpose of the present invention, an invention conceived to solve the problems of prior art described in the foregoing, is to provide an ultrasonic wave surgery apparatus whose operation is intuitive and which can minimize fatigue of the hand.

The purpose of the present invention shall not be limited to the purposes mentioned in the foregoing, and other purposes not mentioned shall be clearly understood by a person having ordinary skill in the art from the following.

### Technical Solution

The ultrasonic wave surgery apparatus controlled by a single switch button of the present invention conceived to achieve the purposes stated in the foregoing comprises: a body with an accommodating space therein, comprising: a grip that can be gripped by the user, and; an ultrasonic wave delivery unit which extends forward by a distance to allow for access to tissue; an transducer unit that is disposed in the accommodating space and which causes oscillations; an ultrasonic transducer unit disposed at the front end of the ultrasonic wave delivery unit and which comprises an end effector that is connected to the transducer unit and receives transmission of oscillations; a gripping unit disposed at the front end of the ultrasonic wave delivery unit and formed to allow variation of the distance thereof from the end effector, comprising an operating portion on the body to allow for control of the gripping portion; a start button disposed on the body and which causes an input signal according to controls by the user, and; a control portion which, if an input signal from the start button occurs once within a preset reference time duration and is sustained, operates the transducer unit at a first oscillation strength, and which, if an input signal from the start button occurs n times within a preset reference time duration and is sustained, operates the transducer unit at an n^{th} oscillation strength.

Further, the start button may be disposed adjacent to the operating portion.

Further, the control portion may operate the transducer unit for the period during which the last unit signal within the reference time duration is sustained.

Also, a guide module which provides information as to the operational state of the transducer unit may further be included.

Further, the guide module may include a display module which indicates the oscillation strength of the transducer unit.

Also, the guide module may include a sounding module which provides information as to the operational state of the transducer unit in the form of sound.

Further, the sounding module may emit different types of sound depending on the oscillation strength of the transducer unit.

Further, the method for controlling the ultrasonic power of the ultrasonic wave surgery apparatus according to the present invention includes a step wherein the control portion of the ultrasonic wave surgery apparatus determines number of input signals caused by manipulating the start button within the preset reference time duration (a), and a step wherein, if an input signal from the start button occurs once within a preset reference time duration and is sustained, the transducer unit is operated at a first oscillation strength, and wherein, if an input signal from the start button occurs n times within a preset reference time duration and is sustained, the transducer unit is operated at an n^{th} oscillation strength (b).

Further, in step (b), the transducer unit may be operated for the duration during which the last input signal within the reference time duration is sustained.

### Benefits of the Invention

The ultrasonic wave surgery apparatus controlled by a single switch button and the control method of ultrasonic power using the same according to the present invention to solve the above-stated problems has the following benefits.

The first advantage is that the control method is intuitive, and can prevent confusion during use.

The second advantage is that as all controls can be effected using a single start button; the button can be disposed near to the gripping portion and operating portion, minimizing fatigue of the hand.

The third advantage is that, as a person carrying out the procedure is informed immediately of the intended operational strength through visual and audio signals, the person carrying out the procedure can concentrate fully on the procedure, thereby carry out the procedure accurately.

The fourth advantage is that, accordingly, procedures can be carried out in a quick and safe manner.

The benefits of the present invention shall not be limited to those benefits mentioned in the foregoing, and a person having ordinary skill in the art shall clearly comprehend other benefits not mentioned from the statements of the claims.

### BRIEF DESCRIPTION OF DRAWINGS

FIG. 1 is a skew drawing depicting the overall appearance of the ultrasonic wave surgery apparatus according to one embodiment of the present invention;

FIG. 2 is a side view of the operating portion of the ultrasonic wave surgery apparatus according to one embodiment of the present invention being manipulated;

FIG. 3 is a skew drawing depicting the gripping of a blood vessel using the gripping portion of the ultrasonic wave surgery apparatus according to one embodiment of the present invention;

FIG. 4 is a side view of the start button of the ultrasonic wave surgery apparatus according to one embodiment of the present invention being pressed one time;

FIG. 5 is a skew drawing depicting the carrying out of blood vessel cauterizing and cutting using the end effector of the ultrasonic wave surgery apparatus according to one embodiment of the present invention;

FIG. 6 is a side view depicting the start button of the ultrasonic wave surgery apparatus according to one embodiment of the present invention being pressed two times within a reference time duration;

FIG. 7 is a skew drawing depicting the carrying out of blood vessel cauterizing using the end effector of the ultrasonic wave surgery apparatus according to one embodiment of the present invention;

FIG. 8 is a skew drawing depicting the appearance of the guide module of the ultrasonic wave surgery apparatus according to one embodiment of the present invention;

FIG. 9 is a drawing showing the action mechanism of the start button of the ultrasonic wave surgery apparatus according to one embodiment of the present invention, and;

FIG. 10 is a drawing showing the action mechanism of the start button of the ultrasonic wave surgery apparatus according to another embodiment of the present invention.

### BEST MODE FOR CARRYING OUT THE INVENTION

In the following, preferable embodiments of the present invention by which the purposes of the present invention may be specifically realized shall be explained with reference to the attached drawings. In explaining these embodiments, the same names and same symbols shall be used for the same components, and additional explanation of such components shall be omitted.

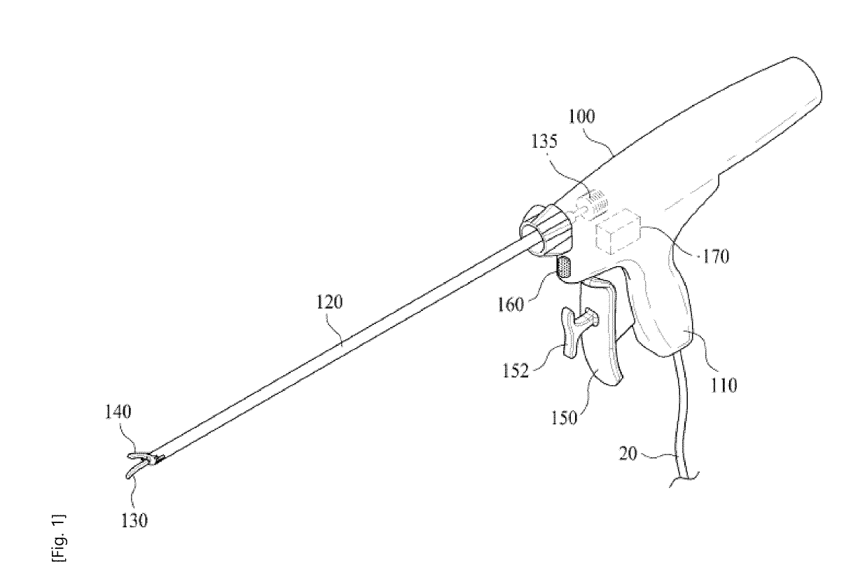

FIG. 1 is a skew drawing depicting the overall appearance of the ultrasonic wave surgery apparatus according to one embodiment of the present invention.

As illustrated in FIG. 1, the ultrasonic wave surgery apparatus according to one embodiment of the present invention comprises: a body (100); an ultrasonic transducer unit comprising a transducer unit (135) and an end effector (130); a gripping unit comprising a gripping portion (140) and an operating portion (150); a start button (160), and; a control portion (170).

An accommodating space is provided within the body (100), and various components may be accommodated within the accommodating space. Further, the body (100) comprises a grip (110) that can be gripped by the user, and an ultrasonic wave delivery unit (120) which extends forward by a distance to allow for access to tissue.

That is, in the case of the present embodiment, the body (100) takes on the overall shape of a gun, allowing for the user to easily grip and use the grip (110). Further, the ultrasonic wave delivery unit (120) is provided so that it can be inserted into the subject receiving the procedure, and may be hollow to accommodate wires and rods to connect the end effector (130) and gripping portion (140) to be explained later to components within the body (100).

The ultrasonic transducer unit is a component provided within the accommodating space of the body (100) and which includes an transducer unit (135) that creates oscillations, and an end effector (130) which is disposed at the front end of the ultrasonic wave delivery unit (120) and connected to the transducer unit (135) to receive transfer of oscillations.

In the present embodiment, the transducer unit (135) is supplied with electrical power from a separate power unit that is connected to a connecting line (20) that extends from the bottom of the grip (110), and causes oscillations according to controls from a control portion (170) to be explained later. Here, the transducer unit (135) and the end effector (130) may be connected to each other by a rod provided within the ultrasonic wave delivery unit (120). Accordingly, the end effector (130) receives transfer of the oscillations caused by the transducer unit (135).

The gripping unit comprises: a gripping portion (140) which is provided at the front end of the ultrasonic wave delivery unit, is formed so that the distance thereof from the end effector (130) can be varied, and which grips tissue together with the end effector (130), and; an operating portion (150) which is provided on the body (100) to allow for manipulation of the gripping portion (140).

In the case of the present invention, the operating portion (150) is provided forward of the grip (110), and is formed to be movable to the front and to the rear, and accordingly the user is able to, while gripping the grip (110), use the operating portion (150) to manipulate the gripping portion (140). As the mechanism for varying the gripping portion (140) by manipulating the operating portion (150) is a matter self-evident to a person having ordinary skill in the art, a detailed explanation will be omitted.

Further, in the case of the present invention, an auxiliary control projection (152) is provided on the front of the operating portion (150) to further facilitate the user's manipulation of the operating portion (150).

The start button (160) is a component provided on the body (100) and which generates input signals according to the user's controls. In the present embodiment, the start button (160) is provided toward the front of the body (100) and near the top of the operating portion (150), according to which the user, while gripping the grip (110), is easily able to manipulate the operating portion (150) and start button (160) using his/her finger.

Further, in the case of the present embodiment, the start button (160) is a press-type button which generates input signals when pressed. Provided, that this is but one embodiment and that various the methods of input are possible for the start button (160).

Also, the method of manipulating the start button (160) shall be explained later.

The control portion (170) is a component that controls the transducer unit (135) according to the input signals generated using the start button (160). That is, when the user presses the start button (160) and generates input signals, the control portion (170) recognizes the same, and operates the transducer unit (135).

Meanwhile, when a single input signal of the start button (160) has been generated, the control portion (170) operates the transducer unit (135) at a first oscillation strength, and if n number of input signals of the start button (160) has been generated with a time difference shorter than the present time duration, operates the transducer unit (135) at an n^{th} oscillation strength.

That is, the control portion (170) determines number of input signals caused by manipulating the start button within the preset reference time duration, and, if an input signal from the start button occurs once within a preset reference time duration and is sustained, operates the transducer unit at a first oscillation strength, and, if an input signal from the start button occurs n times within the preset reference time duration, operates the transducer unit at an n^{th} oscillation strength. Meanwhile, n is a natural number greater than 1, and in the case of the present embodiment, n is 2.

Here, in the present invention, the first oscillation strength is an oscillation strength equal to or greater than a reference oscillation strength, and a second oscillation strength is an oscillation strength less than the reference oscillation strength. Accordingly, in a case wherein an input signal has been generated once within a reference time duration, the oscillation caused will have a strength relatively greater than in a case wherein the input signal has been generated two times within a reference time duration. Provided, that this is but one embodiment, and various settings for the first oscillation strength and the second oscillation strength are possible.

Further, as the start button (160) in the present embodiment is a press-type button, if the user presses the start button (160) one time, the control portion (160) controls the transducer unit (135) so that a strong oscillation at equal to or greater than a reference frequency is generated.

Further, if the user presses the start button (160) 2 times, the control portion (160) controls the transducer unit (135) so that a weak oscillation at less than the reference frequency is generated. Here, the control portion (160) can compute the time difference between the two pressings, and if it is judged that the computed time difference is within the preset reference time duration, it is recognized that the input is of a type different from a single pressing, and the oscillation strength of the transducer unit (135) can be changed.

Here, if a single pressing has been set as strong oscillation, this is generally for the purpose of cauterizing and cutting tissue, and if two pressings has been set to weak oscillation, this is generally for the purpose of cauterizing tissue. In cases where cauterizing is not necessary, the end effector (130) can be placed in contact with the tissue, and the strength of cauterizing or hemostasis can be selected by adjusting strong oscillation and weak oscillation.

As explained in the foregoing, an advantage is provided by the present invention in that usage is easy as the strength/weakness of oscillation for the transducer unit (135) can be controlled using a single start button (160), and in that operation itself is intuitive.

In the following, specific methods of using the ultrasonic wave surgery apparatus according to one embodiment of the present invention will be explained.

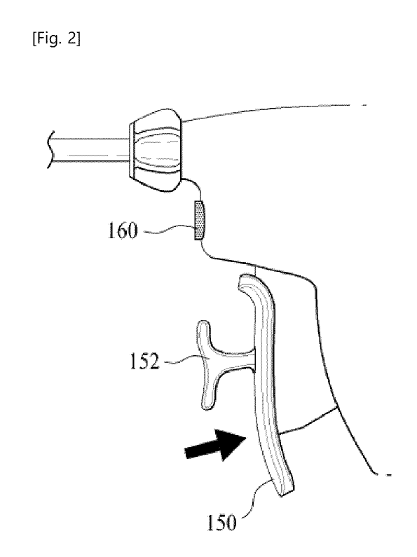

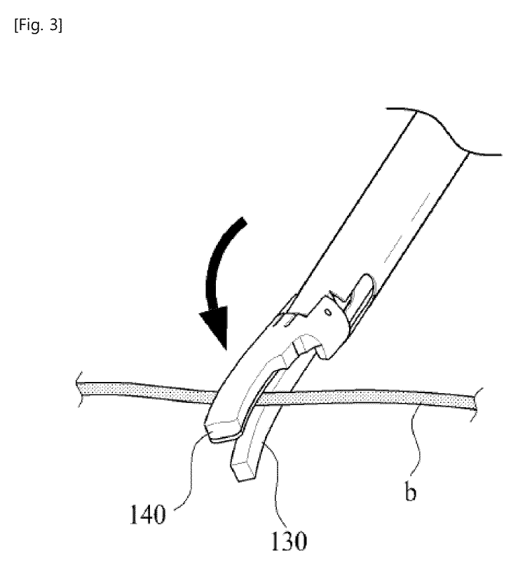

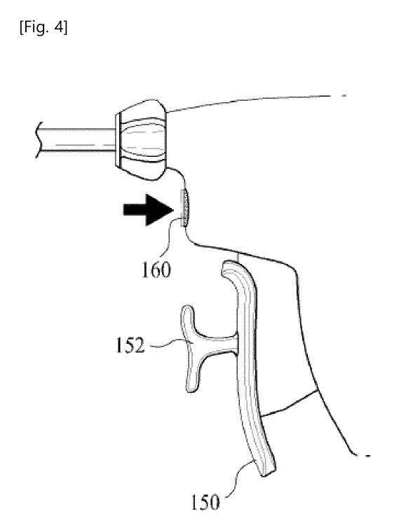

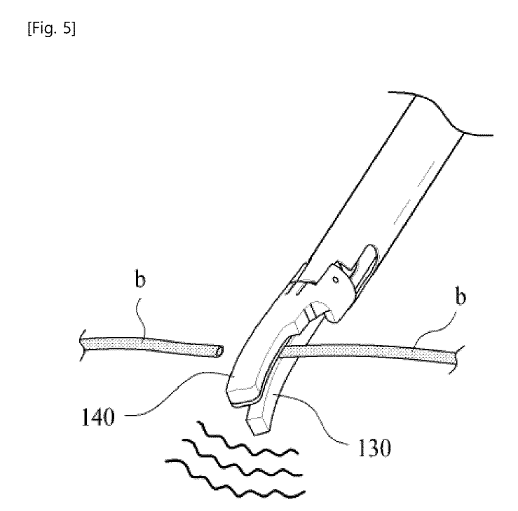

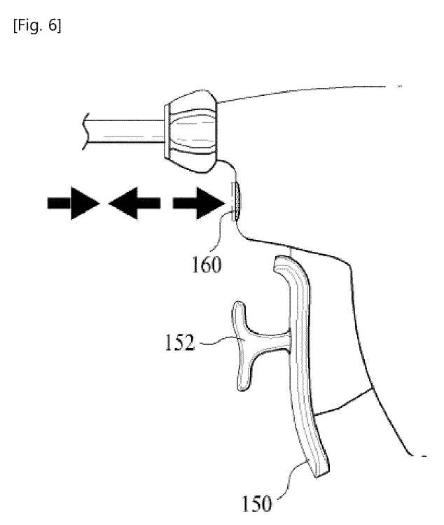

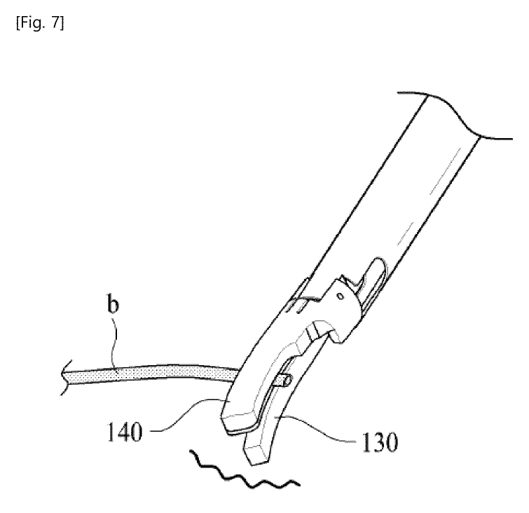

FIG. 2 and FIG. 3 are a side view and skew drawing, respectively, depicting the gripping of a blood vessel (b) using the gripping portion (140) of the ultrasonic wave surgery apparatus according to one embodiment of the present invention. FIG. 4 and FIG. 5 are a side view and skew drawing, respectively, depicting the carrying out cauterizing and cutting of a blood vessel (b) by pressing the start button (160) of the ultrasonic wave surgery apparatus according to one embodiment of the present invention a single time. FIG. 6 and FIG. 7 are a side view and skew drawing, respectively, depicting the carrying out cauterizing of a blood vessel (b) by pressing the start button (160) of the ultrasonic wave surgery apparatus according to one embodiment of the present invention two times.

First, as illustrated in FIG. 2 and FIG. 3, the user places the ultrasonic wave delivery unit of the ultrasonic wave surgery apparatus nearby the tissue of a subject, and positions the tissue between the gripping portion (140) and the end effector (130). In the present embodiment, the tissue is a blood vessel (b).

In this state, the user manipulates the operating portion (150) to move the gripping portion (140) toward the end effector (130), and grips the blood vessel (b).

When intending to carry out cauterizing and cutting of the blood vessel (b), the user, as illustrated in FIG. 4 and FIG. 5, presses the start button (160) one time. Accordingly, the control portion recognizes the input signal and causes the transducer unit to oscillate at an oscillation strength set as the first oscillation strength, with the oscillations caused being transferred to the end effector (130) to carry out cauterizing then cutting of the blood vessel (b).

Further, when intending to carry out cauterizing of the blood vessel (b), the user, as illustrated in FIG. 6 and FIG. 7, presses the start button (160) two times within a reference time duration. Accordingly, the control portion recognizes the input signal and causes the transducer unit to oscillate at an oscillation strength set as the second oscillation strength, with the oscillations caused being transferred to the end effector (130) to carry out cauterizing of the blood vessel (b).

As demonstrated, the user, by using the present invention, is able to intuitively and quickly switch between a first oscillation strength and a second oscillation strength of the transducer unit, and is therefore able to carry out procedures in a quick and safe manner.

Meanwhile, a variety of methods by which the start button (160) is operated are possible, and this will be explained later.

Further, the ultrasonic wave surgery apparatus of the present embodiment may further comprise a guide module (10).

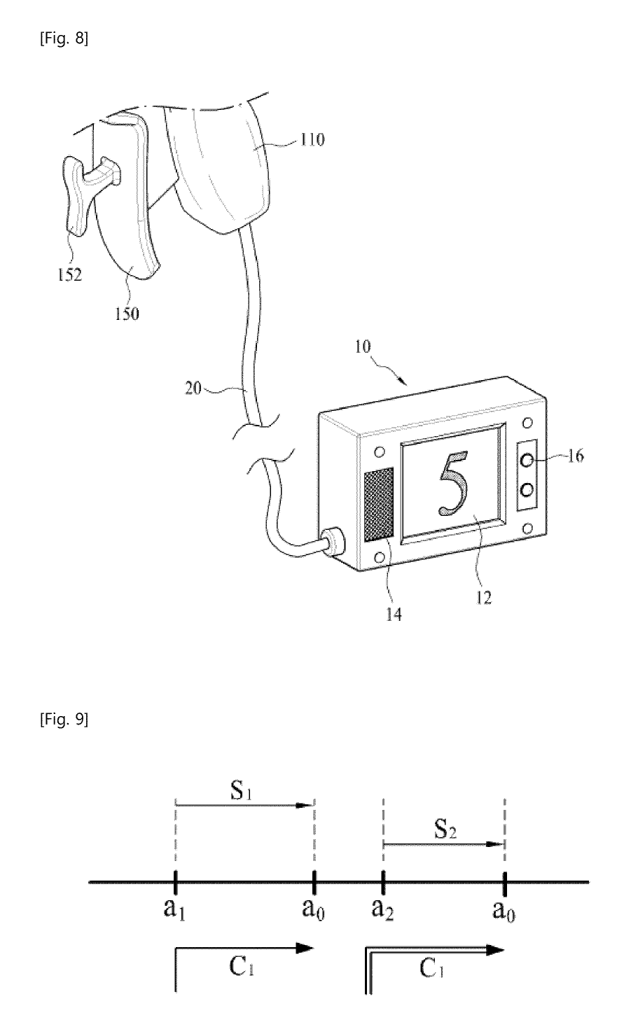

FIG. 8 is a skew drawing depicting the appearance of the guide module of the ultrasonic wave surgery apparatus according to one embodiment of the present invention.

The guide module (10), a component that provides information as to the operational state of the transducer unit, is connected to a connecting line (20) that extends from the bottom of the grip (110) of the ultrasonic wave surgery apparatus in the present embodiment. Here, the guide module (10) may be supplied with electrical power from a separate power unit, and the electrical power may be transferred to the connecting line (20) and be supplied to the body.

Also, the guide module (10) is provided to provide information as to the operational state of the transducer unit in a variety of ways. Specifically, in the present embodiment, the guide module (10) comprises a display module (12) and a sounding module (14).

The display module (12) is a component that visually represents the oscillation strength of the transducer unit according to its oscillation frequency. Whereas, conventionally, a plurality of display modules (12) was provided in many cases to indicate strong and weak oscillations of the transducer unit independently, in the present embodiment, a single module is provided to show the current oscillation strength.

Further, the sounding module (14) is a component that provides information as to the operational state of the transducer unit through sound. That is, it is able to emit sounds to inform that the transducer unit has begun to operate, that the operation strength has been changed, and that operation has stopped. Especially, the present embodiment, different types of sound are emitted depending on the operational frequency of the transducer unit. For example, the sounding module (14) may sound numbers assigned to correspond to oscillation strength levels of the transducer unit, for example ‘Five’ for strong oscillation and ‘Three’ for weak oscillation.

Accordingly, the user, when carrying out procedures, is immediately able to know the operational state without having to gaze upon the guide module (10).

Of course, the sounds emitted by the sounding module (14) may be configured in a variety of other ways.

In the foregoing, the structure and operation of the respective components according to the present invention have been explained. In the following, the action mechanism of the start button will be explained.

The start button, as explained in the foregoing, is able to adjust the oscillation strength of the transducer unit by generating one input signal or two input signals within a reference time duration. This may have a variety of mechanisms.

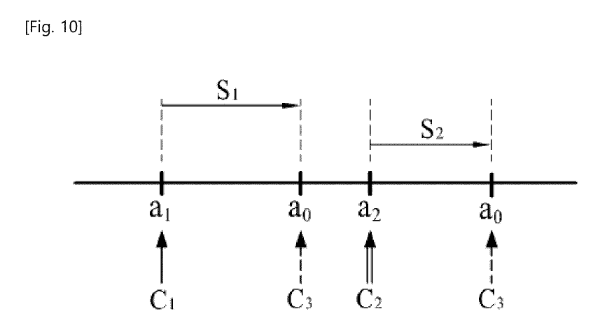

FIG. 9 is a drawing showing the action mechanism of the start button of the ultrasonic wave surgery apparatus according to one embodiment of the present invention, and FIG. 10 is a drawing showing the action mechanism of the start button of the ultrasonic wave surgery apparatus according to another embodiment of the present invention.

In the respective drawings, a₁ is a point in time at which one input signal has been generated, and oscillations of a first oscillation strength (S₁) have been generated, while a2 is a point in time at which two input signals have been generated within a reference time duration, and oscillations of a second oscillation strength (S₂) have been generated. Further, a₀ represents the time in time at which oscillations of a first oscillation strength (S₁) or oscillations of a first oscillation strength (S₂) have ceased. Also, C₁ is a point in time at which the start button has been manipulated one time, and C₂ is a point in time where it has been manipulated two times.

Examining the respective drawings based on this, first, in the case of the embodiment illustrated in FIG. 6, at time point a₁, with the start button having been manipulated one time, oscillations of a first oscillation strength (S₁) begin, while at time point a₂, with the start button having been manipulated two times, oscillations of a second oscillation strength (S₂) begin. Here, by keeping the start button pressed after having generated input signals by pressing, the respective oscillations can be maintained.

That is, the control portion may operate the transducer unit for the duration during which the input signal is sustained.

Next, the case of the embodiment illustrated in FIG. 7 is identical in that, at time point a₁, with the start button having been manipulated one time, oscillations of a first oscillation strength (S₁) begin, and that at time point a₂, with the start button having been manipulated two times, oscillations of a second oscillation strength (S₂) begin. Provided, that in this embodiment, oscillations of the transducer unit are maintained even if the start button is not kept pressed after pressing.

Accordingly, the user's finger is free to move about after pressing [the button], keeping fatigue levels to a minimum.

Further, in this case, a separate signal may be generated to stop the transducer unit. In the present embodiment, operation of the transducer unit is stopped if an input signal occurs at a time point (C₃) after transducer unit operation starts whose time difference [from the start point] exceeds a reference time duration. That is, whereas the control portion recognizes a repeat input signal occurring within a reference time duration as two presses [of the button], if [a second] input signal occurs at a time point beyond a reference time duration [from the first], this can be recognized as a stop command.

As a result, in the present embodiment, the user can stop the oscillations of the transducer unit by pressing the start button again.

Provided, that unlike the respective embodiments, the ultrasonic wave surgery apparatus according to the present invention may, aside from a start button, further include a stop button which, when manipulated by the user, generates a stop signal. In such case, the control portion may start operation of the transducer module at such time a start button input signal has been generated, then stop operation of the transducer module when a stop signal has been generated.

Although the specific exemplary embodiments have been described and illustrated as above, the present invention is not limited to the exemplary embodiments described herein, and it would be apparent to those skilled in the art that various changes and modifications might be made to these exemplary embodiments without departing from the spirit and the scope of the invention. Accordingly, the changed example and modified examples should not be individually appreciated from the technical spirit or the viewpoint of the present invention and it should be appreciated that modified exemplary embodiments will be included in the appended claims of the present invention.

[Explanation of symbols]

| | |
|---|---|
| 10: Guide module | 12: Display module |
| 14: Sounding module | 100: body |
| 110: Grip | 120: Ultrasonic wave delivery unit |
| 130: End effector | 135: Transducer unit |
| 140: Gripping portion | 150: Operating portion |
| 160: Start button | 170: Control portion |
| b: Blood vessel | |

What is claimed is:

1. An ultrasonic wave surgery apparatus controlled by a single switch button, comprising:
a body with an accommodating space therein, comprising: a grip that can be gripped by the user, and;
an ultrasonic wave delivery unit which extends forward by a distance to allow for access to tissue;
an transducer unit that is disposed in the accommodating space and which causes oscillations; an ultrasonic transducer unit disposed at the front end of the ultrasonic wave delivery unit and which comprises an end effector that is connected to the transducer unit and receives transmission of oscillations;
a gripping portion disposed at the front end of the ultrasonic wave delivery unit and formed to allow variation of the distance thereof from the end effector, comprising a control portion on the body to allow for control of the gripping portion;
a start button disposed on the body and which causes an input signal according to controls by the user, and;
a control portion which, if an input signal from the start button occurs once within a preset reference time duration and is sustained, operates the transducer unit at a first oscillation strength, and which, if an input signal from the start button occurs n times within a preset reference time duration and is sustained, operates the transducer unit at an nth oscillation strength.

2. The ultrasonic wave surgery apparatus controlled by a single switch button according to claim 1, wherein the start button is provided adjacent to the operating portion.

3. The ultrasonic wave surgery apparatus controlled by a single switch button according to claim 1, wherein the control portion operates the transducer unit for the duration during which the last input signal within the reference time duration is sustained.

4. The ultrasonic wave surgery apparatus controlled by a single switch button according to claim 1, further comprising a guide module which provides information as to the operational state of the transducer unit.

5. The ultrasonic wave surgery apparatus controlled by a single switch button according to claim 1, wherein the guide module comprises a display module which indicates the oscillation strength of the transducer unit.

6. The ultrasonic wave surgery apparatus controlled by a single switch button according to claim 1, wherein the guide module comprises a sounding module which provides information as to the operational state of the transducer unit in the form of sound.

7. The ultrasonic wave surgery apparatus controlled by a single switch button according to claim 6, wherein the sounding module emits different types of sound depending on the oscillation strength of the transducer unit.

8. A method for controlling the ultrasonic power of the ultrasonic wave surgery apparatus which includes: a step wherein the control portion of the ultrasonic wave surgery apparatus determines number of input signals caused by manipulating the start button within the preset reference time duration (a), and; a step wherein, if an input signal from the start button occurs once within a preset reference time duration and is sustained, the transducer unit is operated at a first oscillation strength, and wherein, if an input signal from the start button occurs n times within a preset reference time duration and is sustained, the transducer unit is operated at an n^{th} oscillation strength (b).

9. The method for controlling the ultrasonic power of the ultrasonic wave surgery apparatus according to claim 8, where, in step (b), the transducer unit is operated for the duration during which the last input signal within the reference time duration is sustained.
